(19) United States
(12) Patent Application Publication
(10) Pub. No.: US 2005/0201952 A1
(43) Pub. Date: Sep. 15, 2005

(54) Treatment and prevention of viral hepatitis infections

(76) Inventor: Yash Sharma, Chantilly, VA (US)

Correspondence Address: BAKER & MCKENZIE LLP, 711 LOUISIANA, SUITE 3400, HOUSTON, TX, 77002-2716, US

(21) Appl. No.: 11/033,812

(22) Filed: Jan. 13, 2005

Related U.S. Application Data
(63) Continuation-in-part of application No. 09/474,677, filed on Dec. 9, 1999.
Application No. 09/474,677 is a continuation-in-part of application No. 09/015,830, filed on Jan. 29, 1998.
(60) Provisional application No. 60/632,982, filed on Dec. 6, 2004.
(60) Provisional application No. 60/114,540, filed on Dec. 29, 1998.

(51) Int. Cl. A61K 9/68; A61K 7/16; A61K 31/739

(52) U.S. Cl. 424/48; 514/54; 424/49

(57) ABSTRACT

Methods for treating a viral hepatitis infection in a subject are described that include administering N-glycolylneuraminic acid or a derivative thereof to the subject.

### CROSS REFERENCE TO RELATED APPLICATIONS

This application claims priority to U.S. Provisional Patent Application No. 60/632,982, filed Dec. 6, 2004, and is a continuation-in-part of U.S. patent application Ser. No. 09/474,677, filed Dec. 29, 1999, which claims priority to U.S. Provisional Application No. 60/114,540, filed Dec. 29, 1998, and is a continuation-in-part of U.S. patent application Ser. No. 09/015,830, filed Jan. 29, 1998, the disclosures of which are incorporated by reference in their entirety.

### BACKGROUND OF THE INVENTION

Hepatitis (inflammation of the liver) is an illness that can cause permanent and life-threatening damage. Hepatitis A, B, C, D, and E viruses may cause viral hepatitis infection. All of these viruses cause short-term and/or acute, viral hepatitis. The hepatitis B, C, and D viruses can also cause chronic hepatitis, in which the infection is prolonged, sometimes lifelong. Other viruses may also cause hepatitis, but they have yet to be isolated and they are obviously rare causes of the disease. Symptoms of viral hepatitis can be debilitating, and they may include: jaundice, fatigue, abdominal pain, loss of appetite, nausea, vomiting, diarrhea, low grade fever, and headache. However, some people having the virus do not have symptoms.

The most common hepatitis viruses are A, B, and C. Hepatitis A virus (HAV) infection is the most common cause of acute hepatitis, and usually resolves spontaneously after several weeks of acute symptoms. Hepatitis B virus (HBV) and hepatitis C virus (HCV) are the most common viral causes of chronic hepatitis, usually defined as liver inflammation persisting for more than six months. Hepatitis B and C infections slowly eat away at a person's liver, severely damaging liver function and greatly increasing the risk of liver cancer. This is relevant because hepatitis, particularly hepatitis C, is involved in about 85 percent of liver cancer cases in the United States. Complications from hepatitis C are blamed for 10,000 deaths per year, but the CDC estimates the fatality rate could triple or quadruple within 15 years.

Hepatitis A is spread primarily through food or water contaminated by feces from an infected person. Rarely, it spreads through contact with infected blood. Hepatitis A can be prevented by the hepatitis A vaccine. Hepatitis B is spread through contact with infected blood, through sex with an infected person, and from mother to child during childbirth. The infection can be prevented by administration of the hepatitis B vaccine. Treatment for hepatitis B infection involves drug therapies.

Hepatitis C virus (HCV) is responsible for the second most common cause of viral hepatitis. Currently, nearly 2% of the U.S. population, and an estimated 170 million people worldwide, are HCV carriers. Hepatitis C is primarily spread through contact with infected blood; less commonly, through sexual contact and childbirth. There is no vaccine for hepatitis C; the only way to prevent the disease is to reduce the risk of exposure to the virus. This means avoiding behaviors like sharing drug needles or sharing personal items like toothbrushes, razors, and nail clippers with an infected person. The only approved therapy for chronic hepatitis C is interferon-alpha (IFN-α), either alone or in combination with ribavirin. Anemia is the most common adverse effect associated with ribavirin treatment and neuropsychiatric adverse effects of IFN-α lead to premature cessation of therapy in 10 to 20% of patients.

Hepatitis D is spread through contact with infected blood. This disease occurs only in people who are already infected with hepatitis B. The people at risk for infection with hepatitis D are those infected with hepatitis B. The most effective means of preventing infection with hepatitis D is immunization against hepatitis B for those not already infected; and avoiding exposure to infected blood, contaminated needles, and an infected person's personal items (e.g., toothbrush, razor, nail clippers, among others). Hepatitis D infection can be treated with alpha interferon.

Hepatitis E is spread through food or water contaminated by feces from an infected person. This disease is uncommon in the United States. There is no vaccine for hepatitis E; the only way to prevent the disease is to reduce the risk of exposure to the virus. This means avoiding tap water when traveling internationally and practicing good hygiene and sanitation. Fortunately, hepatitis E usually resolves on its own over several weeks to months.

Some cases of viral hepatitis cannot be attributed to the hepatitis A, B, C, D, or E viruses. These viruses are called non A-E hepatitis viruses. Scientists continue to study the causes of non A-E hepatitis.

Additional treatment options for viral hepatitis (A-E and non A-E), especially HCV, are urgently needed. Safe, and orally bioavailable anti-hepatitis agents are desirable.

N-glycolylneuraminic acid (Neu5Gc) is a cell surface sialic acid that is immunogenic in humans. Sialic acids, such as Neu5Gc, are N-acyl derivatives of neuraminic acid. Sialic acids occur in many polysaccharides, glycoproteins, and glycolipids in both animals and bacteria. Neu5Gc is widespread throughout the animal kingdom. For example, Neu5Gc is present on the surface of non-human primate cells, as well as, on the surface of baboon cells, goat cells, lamb cells, cow cells and the cells of certain fish, among others. However, due to a genetic mutation in the human gene that hydrolyzes acetyl neuraminic acids, Neu5Gc is nearly absent on the cell surface of human cells.

It has been found that if between about 0.01 mg and 100 mg Neu5Gc/per day is introduced into the human body, stimulation of the human immune system may occur. In response to the introduction of the Neu5Gc, certain antibodies may be produced that could be useful in fighting certain infections and in treating certain immune deficiency diseases (PNAS 100, October 2003)

Attempts have been made to produce Neu5Gc through chemical synthesis, but these efforts have met with limited success. As an alternative to chemical synthesis, Neu5Gc may be produced from biological samples, especially animal tissues. It may be more cost effective to use unpurified or partially purified biological extracts. The Neu5Gc present in certain non-human animals is often found bound to glycoproteins, glycolipids, or other glycoconjugates, or phospholipids. However, in order for Neu5Gc to be more effective in producing an immune response in humans, it may be preferable that it be introduced into the body in a chemically free form. Bound Neu5Gc may be isolated from phospholipids or glycoconjugates using non toxic agents to break its bonds with these compounds.

### SUMMARY OF THE INVENTION

The invention is based on the discovery that N-glycolylneuraminic acid and related compounds can be used to prevent or treat viral infections, as well as other pathogenic infections. N-glycolylneuraminic acid is a complex galactose molecule that is produced in many non-human mammals. N-glycolylneuraminic acid was identified from extracts of baboon peripheral blood monocytes (PBMCs) that were capable of inhibiting HIV-1 replication in and/or infection of human cells. As N-glycolylneuraminic acid is a carbohydrate, toxicity is minimal.

Certain aspects of the present invention are directed to methods for treating a human patient having a viral hepatitis infection with an inflamed liver. The methods involve administering a composition to the patient. The composition administered has at least one of a N-glycolylneuraminic acid or a derivative thereof, and at least one pharmaceutically acceptable excipient. In some aspects, the composition may comprise additional components. The composition may be in the form of a tablet, a lozenge, a sucker, a semi-soft candy, a gum, a gel, a paste, a mouthwash, or a film, in certain aspects of the present invention. The composition is administered in an amount that may be sufficient to reduce inflammation of the patient's liver.

In some aspects of the present invention, the methods may involve determining a baseline hepatitis viral load for the patient prior to administering the composition. The post-administration hepatitis viral load may also be determined. The administration may include a dosing regimen of the composition such that the post-administration hepatitis viral load is less than about 97% of the baseline hepatitis viral load, in certain aspects of the present invention.

Methods of the present invention may be used to treat patients having viral hepatitis infections involving at least one of hepatitis A virus, hepatitis B virus, hepatitis C virus, hepatitis D virus, hepatitis E virus, or a non A-E hepatitis virus. In certain aspects of the present invention, the viral hepatitis infection may be caused by at least a hepatitis C virus.

Other features and advantages of the invention will be apparent from the following detailed description, and from the claims.

### BRIEF DESCRIPTION OF THE DRAWINGS

FIG. 1 is a chromatograph that depicts the HPLC Profile of LUKOR (C18 column; 0.1% TFA/water and a 0-100% ACN gradient; UV detection A280).

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, “buccal administration” refers to oral administration of a composition to a patient that is held in the mouth and is used to deliver N-glycolylneuraminic acid or a derivative thereof into a patient's body. Certain compositions of the present invention may, for example, be held in the patient's mouth and sucked, to release N-glycolylneuraminic acid or a derivative thereof into the buccal cavity.

Regarding “N-glycolylneuraminic acid or derivatives thereof,” N-glycolylneuraminic acid (C₁₁H₁₉NO₁₀, MW 325.3) is the hydroxylated derivative of N-acetylneuraminic acid (sialic acid). As used herein, “derivative” refers to a compound that is similar in structure to N-glycolylneuraminic acid, such as the compounds described in U.S. Pat. Nos. 4,774,326 and 4,774,327 and N-linked glycans, as well as other substituted N-glycolylneuraminic acid compounds. Additional non-limiting examples of derivatives include phosphorylated or sulfated N-glycolylneuraminic acid, N-glycolylneuraminic acid salts, O-glycolylneuraminic acid, as well as other substituted N-glycolylneuraminic acid compounds.

“Bound N-glycolylneuraminic acid or derivatives thereof” refers to N-glycolylneuraminic acid or derivatives thereof that are chemically bound to glycolipids, glycoproteins, other glycoconjugates, or phospholipids.

As used herein, “pharmaceutically acceptable” refers to substances that are generally regarded as safe for introduction into the human body.

An “excipient” refers to an inert substance used in compositions of the present invention to make them easier to administer.

A “therapeutic agent” refers to compounds that are used to treat specific diseases or medical conditions.

“Essential oil” refers to a natural oil with a distinctive scent secreted by the glands of certain aromatic plants having terpenes as the major component. Examples of essential oils include, but are not limited to, citrus oils, flower oils (e.g., rose and jasmine), and oil of cloves.

“Nonnutritive sweetener” refers to a synthetic or natural substance whose sweetness is higher than or comparable to sucrose, and which may have properties such as reduced cariogenicity, health benefits for diabetics, or reduced caloric value compared to sugars.

“Catalytic amount” refers to having a sufficient amount of an acid or salt thereof in a composition of the present invention, such that it may act as a catalyst for hydrolysis. The hydrolysis results in the freeing of at least some N-glycolylneuramic acid or a derivative thereof from being bound to glycoconjugates or phospholipids.

Methods of the present invention are directed to treating a human patient having a viral hepatitis infection. The patient may have an inflamed liver. The methods may involve administering a composition to the patient. The composition may have at least one of a N-glycolylneuraminic acid or a derivative thereof, and at least one pharmaceutically acceptable excipient. Administration of the composition may be sufficient to reduce inflammation of the patient's liver.

In certain methods of the present invention, a baseline hepatitis viral load may be determined for the patient prior to administering the composition. The post-administration hepatitis viral load may also be determined for the patient. In some embodiments, the administration may involving a dosing regimen of the composition such that the post-administration hepatitis viral load is less than about 97% of the baseline hepatitis viral load. The dosing regimen may be such that the post-administration hepatitis viral load is less than about 95% of the baseline hepatitis viral load, in certain embodiments. In some embodiments, the dosing regimen may be such that the post-administration hepatitis viral load is less than about 90% of the baseline hepatitis viral load. Viral load of the subject (i.e., amount of virus/ml of blood) can be determined using polymerase chain reaction (PCR) assays or branched DNA assays to detect copies of viral hepatitis RNA.

In certain methods of the present invention, at least one liver function test that produces results outside of the normal range prior to administering the composition can be done. Following administration of a composition of the present invention, the same liver function test(s) are done post-administration. The administration can involve a dosing regimen of the composition such that results of at least one post-administration liver function test is at least closer to the normal range than the liver function test performed prior to administering the composition. In some embodiments, a post-administration liver function test finds that the patient has results in the normal range.

Tests that can be used to assess liver function and damage are discussed below. AST or SGOT (serum glutamic-oxaloacetic transaminase) is an enzyme released by the liver, heart muscle, skeletal muscle, pancreas and kidneys into the blood stream when disease or injury to these organs occurs. A clinical diagnosis of liver damage cannot be based on this lab value alone, since several organs contain this enzyme. Normal AST values are 9-34 IU/liter. ALT or SGPT (serum glutamic-pyruvic transaminase) is an enzyme found in high concentrations in the liver and at lower concentrations in the heart, kidney and skeletal muscle. Both SGOT and SGPT are found in relatively low numbers in the peripheral blood. When elevations do occur in both of these enzymes, it usually indicates that some tissue destruction of the liver is occurring. Normal SGPT values are between 6 and 41 IU/liter. Total bilirubin (T Bili) can also be tested. Bilirubin is a waste product of the breakdown of old or damaged red blood cells. Bilirubin normally enters the blood stream and circulates until it reaches the liver and bowel where it is further broken down and excreted. Normal values for T Bili in serum are between 0.1 and 1.5 mg/dliter. Bilirubin is not normally found in the urine. ALP (alkaline phosphatase) is an enzyme found in bone, liver, kidney, intestine, and placenta. During bone formation or with liver and biliary tract disorders, serum levels will rise in proportion to the severity of the condition. Normal values for ALP are between 37 and 116 IU/liter. In chronic hepatitis C, the ALT and AST levels range from 0 to 20 times the upper limit of normal. Gamma glutamyl transpeptidase (GGT) can also be measured and is considered to be normal between 0 and 51 IU/liter. Prothrombin time (PT) can also be determined, and is normal if between 11 to 13.5 seconds. Finally, albumin levels can be determined and the normal range is between 3.4 to 5.4 g/dliter. Viral hepatitis can cause at least certain test values for a patient to fall outside the normal ranges discussed above.

Certain methods of the present invention may involve administering the composition having at least one of a N-glycolylneuraminic acid or a derivative thereof in addition to at least one of an antiviral medication, a medication to alleviate or reduce a symptom of the hepatitis infection, or a medication to alleviate or reduce a side effect of a drug being administered to the patient. In some methods of the present invention, the composition is administered as a prophylactic to the patient, and the method involves administering the composition to the patient in an amount sufficient to prevent viral hepatitis infection.

In certain aspects of the invention, the viral hepatitis infection may involve at least one of hepatitis A virus, hepatitis B virus, hepatitis C virus, hepatitis D virus, hepatitis E virus, or a non A-E hepatitis virus. The viral hepatitis infection may involve hepatitis C virus, in certain embodiments.

In some embodiments, the composition may have between about 0.002 wt % and 20 wt % of the N-glycolylneuraminic acid or the derivative thereof. In other embodiments, the composition may have between about 0.1 wt % and 20 wt % of the N-glycolylneuraminic acid or the derivative thereof, and in some, between about 0.1 wt % and 10 wt % of the N-glycolylneuraminic acid or the derivative thereof.

In certain embodiments, the composition may have a derivative of N-glycolylneuraminic acid, and the derivative may be a phosphorylated N-glycolylneuraminic acid or a sulfated N-glycolylneuraminic acid, among others known in the art. In certain aspects of the present invention the composition may have synthetic N-glycolylneuraminic acid or a derivative thereof. The composition may have bound or free N-glycolylneuraminic acid or a derivative thereof that has been extracted from a biological sample in some aspects of the invention. In certain embodiments, the composition may have a biological sample that comprises the N-glycolylneuraminic acid or a derivative. In particular, N-glycolylneuraminic acid may be obtained from or be a component of at least one of a sea cucumber extract; a peripheral blood mononuclear cell extract of a non-human animal (e.g., a pig or baboon PBMC extract); a submaxillary gland extract of a non-human animal (e.g., a equine, bovine or porcine submaxillary gland extract); an extract from meat or meat fat (e.g., beef, pork, lamb, and poultry) consumed by humans; a milk, butter or cheese extract (e.g., bovine, goat, and sheep milks and cheeses); a fish extract (e.g., cod, tuna, and salmon extracts), a starfish extract, a shark extract, a crocodile extract, or a sea urchin extract, among others known in the art. In certain aspects of the present invention, a sea cucumber extract, a pig submaxillary gland extract, a salmon extract, a milk, butter, or cheese extract, a lamb extract, a pork extract, a beef extract, or a beef fat extract may be used as the source of bound or free N-glycolylneuraminic acid or derivatives thereof in the composition. In some embodiments of the present invention, a sea cucumber extract, a meat extract, a meat fat extract, or a goat cheese extract may be used. In some aspects of the present invention, a sea cucumber extract may be used. Extracts comprising N-glycolylneuraminic acid or derivatives thereof may be purchased commercially from, for example, Sigma Aldrich as a porcine submaxillary gland extract. (PNAS 100, October 2003)

In some embodiments, the composition that is administered to patient having a viral hepatitis infection may have at least some N-glycolylneuraminic acid or derivative thereof that is bound, and the composition may further have a catalytic amount of at least one pharmaceutically acceptable acid or a salt thereof. Certain compositions of the present invention may have between about 0.01 wt % and 50 wt % of an acid or a salt thereof. In certain aspects of the present invention, the compositions may have between about 0.01 wt % and 5 wt % of an acid or a salt thereof. In some embodiments, the compositions may have between about 0.1 wt % and 5 wt % of an acid or a salt thereof. Examples of acids that may be used in the present invention include: salicylic acid, glycolic acid, phosphoric acid, pentathoic acid, and ascorbic acid, among others known in the art. The composition may comprise salts of such acids.

In certain aspects, the composition comprises ascorbic acid or salt thereof. Examples of ascorbic acid salts that could be used in compositions of the present invention include mono-, di-,and tri-sodium citrate salts of ascorbic acid, among others. Such acids or salts thereof are available commercially from Sigma Aldrich, St. Louis, Mo. Certain compositions of the present invention may comprise between about 0.002 wt % and 20 wt % ascorbic acid or a salt thereof. In some embodiments, the compositions comprise between about 0.01 wt % and 5 wt % ascorbic acid or a salt thereof. In certain embodiments, the compositions comprise between about 0.1 wt % and 5 wt % ascorbic acid or a salt thereof.

Compositions employed in the present invention may have a pharmaceutically acceptable excipient and may be, for example, mannitol, cyclodextrins and their derivatives magnesium stearate, calcium carbonate, sodium carbonate, lactose, D-mannitol, calcium phosphate, sucrose, sodium chloride, glucose, starch, kaolin, cellulosic materials, anhydrous calcium secondary phosphate, light anhydrous silicic acid, partly pregelatinized starch, acacia powder, gum arabic, sorbitol, corn starch, or alginic acid, among others known in the art. In some embodiments other components listed below may be used as an excipient (e.g., a lubricant such as magnesium stearate).

According to some embodiments, the excipient may serve more than one role in the composition. For example, mannitol may function as both a nonnutritive sweetener and an excipient. Similarly, the excipient may serve as a flavorant, buffering agent, lubricant, or other component of the composition. The excipient may be present in an amount less than about 90 wt % by weight (wt %), in some embodiments in an amount less than about 80 wt %, and in certain embodiments, in an amount less than about 50 wt % in a composition of the present invention.

Compositions of the present invention may be administered by a number of different methods known in the art. For example, the composition may be administered intravenously, subcutaneously, by inhalation, transdermally, or buccally. In certain embodiments in which a composition is administered buccally, the composition may be at least partially dissolved by saliva in the patient's mouth. In some embodiments involving buccal administration, at least some of the N-glycolylneuraminic acid or derivative thereof may be bound, and the composition may have a catalytic amount of at least one pharmaceutically acceptable acid or a salt thereof, and at least some of the acid or salt thereof may be dissolved by saliva in the patient's mouth. In certain embodiments, compositions for buccal administration of the present invention may have at least one extract having at least one bound N-glycolylneuraminic acid or a derivative thereof, at least one of ascorbic acid or a salt thereof, and at least one pharmaceutically acceptable excipient. In certain embodiments, the composition may have between about 0.002 wt % and 20 wt % bound N-glycolylneuraminic acid or a derivative thereof, and between about 0.02 wt % and 50 wt % ascorbic acid or a salt thereof.

The composition may be in the form of a tablet, a lozenge, a sucker, a semi-soft candy, a gum, a gel, a paste, a mouthwash, or a film, in certain embodiments of the present invention. In some embodiments, compositions of the present invention may further have at least one pharmaceutically acceptable component selected from a coloring agent, a polypeptide, a lubricant, a coating, a sweetener, a flavoring, an antibacterial agent, a taste modifier, a preservative, a disintegrator, a disintegration-preventor, a binder, an antioxidant, a dietary supplement, an antiblocking agent, an antisticking agent, an absorption promoter, absorption-adsorption carriers or a therapeutic agent, among others known in art. Such components when present in certain embodiments do not interfere with release and absorption of N-glycolylneuraminic acid. As with the excipient, some of these components of a composition may serve in more than one role.

Examples of disintegrators, include dry starch, alginic acid, agar powder, crosslinked polyvinyl pyrrolidone, crosslinked sodium carboxymethylcellulose, L-hydroxypropylcellulose, calcium carboxymethylcellulose, and sodium starch glycolate, among others. Examples of disintegration-preventors, are stearyl alcohol, stearic acid, cacao butter, and hydrogenated oil, among others. Binders such as gelatin, crystalline cellulose, simple syrup, sucrose, glucose solution, starch solution, polyvinyl alcohol, polyvinyl ether, polyvinylpyrrolidone, carboxymethylcellulose, shellac, methylcellulose, ethylcellulose, sodium alginate, gum arabic, hydroxypropylmethylcellulose, hydroxypropylcellulose, water, D-mannitol, dextrin, ethanol, starch, gelatin, and acacia, among others may be used in certain compositions.

Antiblocking and antisticking agents such as aluminum silicate, calcium hydrogen phosphate, magnesium oxide, talc, and silicic acid anhydride, among others, may be used in certain compositions of the present invention. Lubricants such as magnesium stearate, calcium stearate, stearic acid, camauba wax, light salicylic acid anhydride, aluminum silicate, magnesium silicate, hardened oil, hardened vegetable oil derivatives, colloidal silica, sesame oil, bleached bees wax, titanium oxide, dry aluminum hydroxide gel, calcium hydrogen phosphate, sodium lauryl sulfate, polyethylene glycol, and talc, among others, may be used in certain compositions of the present invention. In certain embodiments, the lubricant may be present in an amount between about 0.1 and 25 wt %, in certain embodiments in an amount between about 0.1 and 10 wt %, and in certain aspects of the invention in an amount between about 0.1 and 5 wt % of the inventive composition. Examples of absorption promoters that may be used in certain embodiments of the present invention include quaternary ammonium salts, sodium lauryl sulfate, urea, and enzymes, among others. Examples of absorption-adsorption carriers are starch, lactose, kaolin, bentonite, silicic acid anhydride, hydrated silicon dioxide, magnesium metasilicate-aluminate, and colloidal silicic acid, among others.

Further, if desired, a tablet, a semi-soft candy, a gum, a sucker, or a lozenge may be coated. The coating may be made with sugar, or gelatin, among others compounds. The coating may comprise hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudragit (methacrylic acid/acrylic acid copolymer, manufactured by Rohm and Haas, DE), or pigment (e.g., iron oxide red, titanium dioxide, et.), among other coating components known in the art.

Flavorings that may be used in the present invention include sweeteners, especially non-nutritive sweeteners. Examples of flavorings that could be used in certain embodiments, include acacia or tragacanth, among others. Certain compositions of the present invention may comprise more than one sweetener. In certain embodiments, the flavoring comprises a nonnutritive sweetener that is noncariogenic. The cariogenicity of a substance is dependent upon its susceptibility to fermentation by Streptococcus mutans and other oral microorganisms. Dental researchers have long recognized that fermentable sweeteners such as sucrose, glucose, starch, and corn syrup are cariogenic or cavity causing. Examples of nonnutritive sweeteners that may be used in compositions of the present invention include: saccharin, invert sugar, cyclamate, palantinose, aspartame, xylitol, acesulfame, sorbitol, monellin, mannitol, meohesperidine, maltitol, and palatinit, among others. In certain compositions of the present invention, the nonnutritive sweetener may be present in an amount between about 50 and 90 wt %, in certain embodiments in an amount between about 70 and 90 wt %, and in some embodiments in an amount between about 80 and 90 wt %.

Other flavorings that may be used in compositions of the present invention include a candy taste, such as chocolate, orange, vanilla, and the like; essential oils such as peppermint, spearmint and the like; or other flavor, such as anis seed, eucalyptus, 1-menthol, carvone, and anethole, among others known in the art. Both individual and mixed flavors are contemplated. The flavorings are generally utilized in amounts that will vary depending upon the individual flavor, and may, for example, range in amounts of about 0.1% to about 6% by weight of the final composition.

In certain embodiments, fluoride, and more particularly sodium monofluorophosphate or sodium fluoride may be incorporated into a composition of the present invention, especially one having a nonnutritive sweetener, such as xylitol.

The coloring agents useful in the present invention include pigments which may be incorporated in amounts of up to about 2% by weight of the composition. Also, the coloring agents may include other dyes suitable for food, drug and cosmetic applications (i.e., FD&C dyes) and the like. The materials acceptable for the foregoing spectrum of use are, in some embodiments, water-soluble. Illustrative examples include the indigo dye known as FD&C Blue No. 2, which is the disodium salt of 5,5-indigotindisulfonic acid, FD&C Green No. 1, which is a triphenylmethane dye and is the monosodium salt of 4-[4-N-ethyl-p-sulfobenzyl amino)diphenyl-methylene]-[1-(N-ethyl-N-p-sulfoniumbenzyl)-2,5-cyclohexadienimine]. Other FD&C and D&C colorants useful in the present invention and their corresponding chemical structures may be found in the Kirk-Othmer Encyclopedia of Chemical Technology, 3rd Edition, in Volume 6, at pages 561-595.

The amount of N-glycolylneuraminic acid or a derivative thereof effective for preventing or treating a viral infection in a subject may vary, depending on a number of factors, including the amount of the composition in individually prepared doses (e.g., tablets) conveniently available, the chemical characteristics of the compounds employed, the formulation of the compound excipients and the route of administration. The optimal dosage of N-glycolylneuraminic acid to be administered also may depend on such variables as the overall health status of the particular patient and the relative biological efficacy of the compound (e.g., N-glycolylneuraminic acid or derivatives thereof) selected. Compositions of the present invention may have between about 0.002 wt % and 20 wt % bound or free N-glycolylneuraminic acid or a derivative thereof. In some embodiments, the compositions may have between about 0.01 wt % and 5 wt % bound or free N-glycolylneuraminic acid or a derivative thereof. In certain embodiments, the compositions may have between about 0.01 wt % and 2.5 wt % bound or free N-glycolylneuraminic acid or a derivative thereof.

Dosing regimens that may be used in the present invention include administering the composition at least once per day. In certain embodiments, the dosing regimen involves administering the composition at least three times per day. In some embodiments, the dosing regimen involves administering the composition at least four times per day. In addition, delayed release formulations of the composition may be used such that administrations are less frequent. In certain embodiments, a physician may prescribe the proper dosages and dosing regimen. The composition may be, in certain embodiments, in unit dosage form. In such form the composition is subdivided into unit doses containing appropriate quantities of the active component. The unit dosage form may be a packaged preparation, the package containing discrete quantities of preparation, such as packeted tablets or lozenges. In certain embodiments, the unit dosage form may be a tablet or lozenge itself, or it may be the appropriate number of any of these in packaged form.

The dosing regimen in certain aspects of the present invention may involve administering to the patient between about 0.1 mg and 1000 mg of the composition per day. In some embodiments, between about 0.2 mg and 100 mg of the composition may be administered per day, and in certain embodiments, between about 0.2 mg and 80 mg of the composition may be administered per day.

### EXAMPLES

### Example 1

### Isolation of a Small Molecule from Baboon Blood

Peripheral blood monocytes (PBMC) were isolated from whole baboon blood using Ficoll-Hypaque density gradient centrifugation or from PBMCs further expanded in tissue culture following activation with phytohemagglutinin -P (PHA-P) and growth in medium containing interleukin-2 (IL-2). In either case, the PBMCs first were washed 3 times with sterile phosphate-buffered saline (PBS) and pelleted by centrifugation. The cell pellet then was lysed by resuspension in sterile H₂O and held for 96 hours at 4° C. Proteins and nucleic acids were precipitated from the extract and the remaining components in the extract were stabilized using 10% (v/v) calcium phosphate buffer (pH 7.4) containing 0.01% calcium chloride and 0.001% ascorbic acid. The solution was clarified by centrifugation followed by filtration through a 0.22 μm filter. This final filtrate represented a 1:50 dilution of the initial cell lysate and is hereafter referred to as LUKOR. In some instances, the LUKOR preparation was sterilized by Cobalt radiation at 2.5 mRADs for 3 hours (Neutron Products, Inc. Gaithersburg, Md.).

A series of in vitro and in vivo toxicology studies were conducted to evaluate the potential adverse effects of LUKOR on human blood cells, blood clotting factors, and in rats administered intravenous LUKOR repeatedly over 28 days. Freshly drawn heparinized whole blood (1 ml) was diluted 1:4 in PBS (pH 7.4) and 0.1 ml of LUKOR (freshly prepared) was added per ml of diluted whole blood. Two aliquots of the blood were maintained at 4° C. for 21 and 42 days. Extract was not added to the control samples (freshly drawn blood, and blood stored for 21 and 42 days). Results are reported in Table 1.

TABLE 1
Addition of LUKOR to whole blood

| Param- | Day O | | Day 21 | | Day 42 | |
|---|---|---|---|---|---|---|
| eter | Untreated | Treated | Untreated | Treated | Untreated | Treated |
| RBC | 5.3 | 5.3 | 4.2 | 5.1 | 3.0 | 5.1 |
| Hb | 12.8 | 12.9 | 8.4 | 12.8 | 6.3 | 12.6 |
| Hct | 32.3% | 32.3% | 20.2% | 33.0% | 15.8% | 32.0% |
| Lysis | 0% | 0% | 10% | 0% | 40% | 0% |
| 45% | 0% | 0% | 92% | 0% | 58.4% | 0% |
| saline | | | | | | |

The effects of LUKOR on Factor VIII and Factor IX clotting activities were determined by adding LUKOR (1:10 dilution) to human plasma and incubating the samples for 1, 2, 5, and 23 hours at 37° C. The samples were frozen and assayed for Factor VIII and Factor IX clotting activity by a one-stage activated prothrombin time (APTT) method using 0.15 M sodium chloride as the control. Denson, Br. J. Haematol., 1973, 24(4):451-461. Samples also were incubated at room temperature and at 4° C. Factor VIII and Factor IX activities of the samples treated with LUKOR (0.076 and 0.389 units/ml respectively) were not different from saline controls (Table 2, % activity remaining).

TABLE 2
Clotting Activity in Treated Samples

| Time (Hrs) | FVIII/saline | FVIII/LUKOR | FIX/Saline | FIX/LUKOR |
|---|---|---|---|---|
| 0 | 91.81 | 111.11 | 91.13 | 93.01 |
| 1 | 93.92 | 114.29 | 98.77 | 103.06 |
| 2 | 80.11 | 120.91 | 96.95 | 93.83 |
| 5 | 93.5 | 114.29 | 93.51 | 93.68 |
| 23 | 67.95 | 87.45 | 40.14 | 62.03 |

In vivo toxicology of LUKOR was examined using Sprague-Dawley CD albino rats. Five male rats and five female rats were administered 1 ml of LUKOR per day by intravenous injection into the tail vein on 6 occasions (on Days 1, 3, 5, 7, 9 and 28). All animals were examined twice daily for mortality and signs of ill health or reaction to treatment, with a more detailed examination performed weekly. There were no treatment-related clinical signs and the animals did not develop hypersensitivity reactions by the end of the experiment on Day 28. No treatment-related effects on food consumption or weight gain were observed. Blood samples were collected under anesthesia from the orbital sinus, 24 hours following the dose at day 9, and again from the abdominal aorta at necropsy at day 28. All animals were euthanized by exsanguinations from the abdominal aorta following anesthesia. At the termination of the study on Day 28, hematological evaluations were performed on all animals. There were no mortalities, no treatment-related clinical observations, or effects on white cell parameters.

### Example 2

### Cytotoxicity of LUKOR on Cultured Blood Mononuclear Cells

Cultured human blood mononuclear cells were cultured in the presence of varying concentrations of LUKOR for 7 days. Solutions containing different concentrations of LUKOR were prepared by diluting the stock solution of LUKOR (1 mg/mL) 1:4, 1:20, 1:100, and 1:500 with PBS. An equal volume of each dilution of LUKOR was added to cultured cells. Medium was changed at Day 3. The resulting cell counts at each concentration of LUKOR are listed in Table 3. A colorimetric assay was used to assess cytotoxicity. A WST-1 test kit (a tetrazolium compound that is the sodium salt of 4-[3-(4-iodophenyl) -2-(4-nitrophenyl)-2H-5-tetra olio]-1,3-benzene dislocate) was used in this assay (Roche Diagnostics, Indianapolis, Ind.).

TABLE 3
Cytotoxicity of LUKOR

| Dilution (1×) | WST-1 (%) | Cell Count (%) |
|---|---|---|
| 0 | 100 | 100 |
| 4 | 84 | 78.8 |
| 20 | 98 | 106 |
| 100 | 101 | 100 |
| 500 | 104 | 106 |

### Example 3

### Identification of Active Component from LUKOR

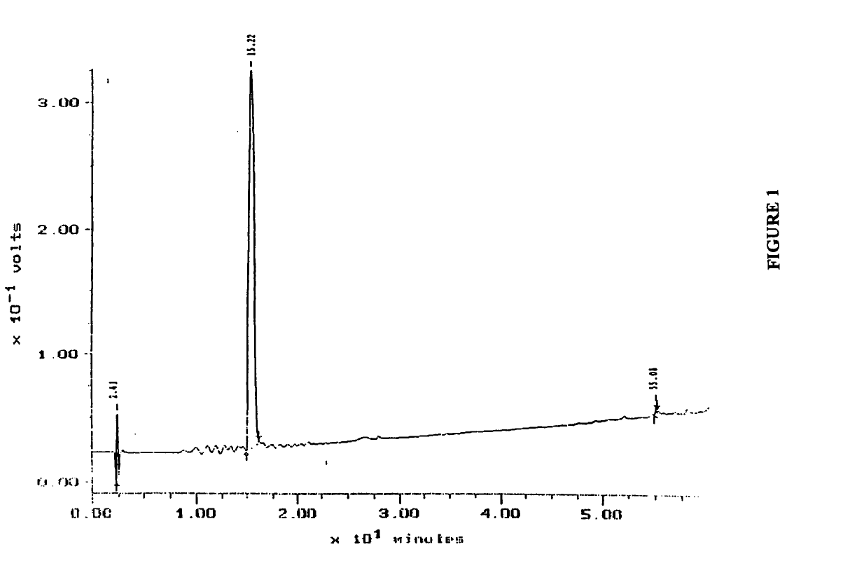

The soluble lysate isolated from PBMCs (LUKOR) was fractionated by HULK as a first step in the identification of the active component. A C18 column (Delta Pak, 15 μm, 300 Å, 0.39×30 cm) with a mobile phase of 0.1% tetrafluoroacetic acid (TFA) in water and a gradient of 0-100% acetonitrile (ACN) was used to separate the components in the cell lysate. One major peak eluting with 30% ACN and two minor peaks at 50% ACN were observed (FIG. 1). The 3 peaks, designated HPLC-1, HPLC-2, and HPLC-3, were collected separately and lyophilized and stored for further characterization by mass spectrometry and NMR.

Mass spectrometry was performed with a VG BioQ triple quadrupole mass spectrometer operating in the positive ion electrospray ionization mode using the following parameters: scan range m/z 100-950 and 35-700; cone voltage 57V to 63V; source temperature 80° C. to 100° C. Calibration was performed with direction injection analysis of CsI prior to LC-MS. A distinct aromatic ring absorbance with a peak maximum at 258 nm was detected. Behavior of the compound was consistent with a small molecular weight compound. Sample related masses of 86, 194, and 288 were identified. The sample related masses of 86 and 194 represented less than 1% of total. It was determined that the sample mass of 288 was composed of carbon, hydrogen, oxygen, and a single nitrogen atom.

Proton NMR also was performed on the extract in a solution of D₂O using a modified Nicolet NT 360 MH3 spectrometer operating with a single 0.5μ sec excitation pulse and a one second re-cycle delay. Signals were detected with shifts between 3.1-3.7 ppm.

The active component of LUKOR was identified as N-glycolylneuraminic acid based on the molecular weight and chemical composition.

### Example 4

### Assessment of Cell Proliferation in Cells Treated with N-glycolylneuraminic acid

On day 0, cell lines listed in Table 4 were plated into microtiter plates at 850-2000 cells/well in 100 μL of media. On day 1, N-glycolylneuraminic acid was diluted 2× in medium and from 0 to 200 μM of N-glycolylneuraminic acid or 1000 μM of N-glycolylneuraminic acid were added. The stock solution at 50/250 mM in DMSO then was diluted 1/500 in media, V_{f}=200 μL/well. The cells were incubated for 3 days at 37° C. and 5% CO₂. On day 4, ³[H]-thymidine, diluted 1/100 in media, was added at 25 μL/well/200 μL of medium, resulting in a final concentration of 0.5 μCi per well. On day 5, cells were harvested (18 hours after the addition of the ³[H]-thymidine) onto a glass fiber, and CPM/ well were determined. The results are listed in Table 4.

TABLE 4
Treated Cell Lines

| | | Plating | |
|---|---|---|---|
| | | cpm | % Confluency |
| Cell Line | Description | (day 0) | (day 5) |
| Molt-4 | Peripheral blood, acute | 2000 | 50 |
| | lymphoblastic leukemia,human. | | |
| DU-145 | Prostate carcinoma, metastasis to | 1200 | 100 |
| | brain, human | | |
| HSF | Human foreskin fibroblast, | 1000 | 100 |
| | diploid, normal. | | |
| HT1080 | Fibrosarcoma, epithelial-like, | 850 | 100 |
| | human. | | |
| HepG2 | Hepatocellular carcinoma, human. | 2000 | 50 |

### Example 5

### Inhibition of Tumor Necrosis Factor (TNFα) with N-Glycolylneuraminic Acid

TNFα production was examined in latently infected with HIV promonocytic U1 cells that were incubated in the presence of N-glycolylneuraminic acid (0.1 to 316 μm). TNFα production was determined by an enzyme linked immunosorbent assay (ELISA) in a competitive format, using a commercially available anti-TNFα antibody (Sigma). Inhibition of TNFα production is reported as percent of saline control in Table 5.

TABLE 5
Inhibition of TNFα Production in U1 Cells

| Conc. of N-Glycolylneuraminic Acid | Inhibition of TNFα Production |
|---|---|
| (μM) | % of Control |
| 0.1 | 0 |
| 0.3 | 0 |
| 10 | 9.8 |
| 30 | 22.6 |
| 100 | 96 |

### Example 6

### Inhibition of Viral Hepatitis

Lozenges were manufactured by two companies, D&E pharmaceuticals, NJ, and Edge Labs, NJ. The lozenges comprised sea cucumber extract available from Global Nutrients, NJ. All the lozenges were prepared from a single lot of sea cucumber extract. The lozenges from both manufacturers comprised N-glycolylneuraminic acid and/or derivatives thereof, and sodium ascorbate. The lozenges also contained magnesium stearate. Lozenges were analyzed for content by Eurofins, Petaluma, Calif. (See Table 6 below for analysis of the two tablets.)

TABLE 6

| | Total weight (mg) | N-glycolylneuraminic | |
|---|---|---|---|
| Sample | of tablet | acid + derivatives (mg) | Ascorbate (mg) |
| 1 | 500 | 0.375 | 0.22 |
| 2 | 500 | 0.22 | 1.52 |

Patients were administered 2 lozenges 3 times per day for 42 days. The patients were instructed to hold lozenge in the mouth until completely dissolved for 2 to 60 minutes. The patients were co-infected with HIV and hepatitis C.

The patients' viral load values, were evaluated. (See Table 7.) The first draw was at 30 days and the second draw was at 60 days.

TABLE 7

| | | | Second Draw |
|---|---|---|---|
| | Baseline | First Draw | Hepatitis C Viral |
| Patient | Hepatitis Viral Load | Hepatitis Viral Load | Load |
| 1 | 9,188,540 | 4,181,210 | 5,730,000 |
| 2 | 7,315,000 | 7,272,000 | 7,070,000 |
| 3 | 6,465,070 | 284,773 | 1,508,910 |
| 4 | 6,302,360 | 2,139,900 | 2,950,000 |
| 5 | 2,237,540 | 1,106,220 | 6,750,000 |
| 6 | 8,358,520 | 6,678,290 | Not taken |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a human patient having a viral hepatitis infection with an inflamed liver comprising,
administering to said patient a composition comprising,
at least one of a N-glycolylneuraminic acid or a derivative thereof, and
at least one pharmaceutically acceptable excipient, wherein the administration of the composition is sufficient to reduce inflammation of the patient's liver.

2. The method of claim 1, further comprising the steps of determining a baseline hepatitis viral load for the patient prior to administering the composition, and determining a post-administration hepatitis viral load for the patient, wherein the administration comprises a dosing regimen of the composition such that the post-administration hepatitis viral load is less than about 97% of the baseline hepatitis viral load.

3. The method of claim 1, wherein the composition is administered in a dosing regimen such that the post-administration hepatitis viral load is less than about 95% of the baseline hepatitis viral load.

4. The method of claim 1, wherein the composition is administered in a dosing regimen such that the post-administration hepatitis viral load is less than about 90% of the baseline hepatitis viral load.

5. The method of claim 1, further comprising the steps of
performing at least one liver function test that produces results outside of the normal range prior to administering the composition, and
performing the at least one liver function test post-administration, wherein
administration comprises a dosing regimen of the composition such that results of the post-administration liver function test are at least closer to the normal range than the liver function test performed prior to administering the composition.

6. The method of claim 5, wherein the results of the post-administration liver function test are within the normal range.

7. The method of claim 1, wherein at least some of the N-glycolylneuraminic acid or derivative thereof is bound, and the composition further comprises a catalytic amount of at least one pharmaceutically acceptable acid or a salt thereof.

8. The method of claim 1, wherein the composition is administered intravenously, subcutaneously, by inhalation, transdermally, or bucally.

9. The method of claim 1, wherein the composition is administered buccally.

10. The method of claim 9, wherein the composition is at least partially dissolved by saliva in the patient's mouth.

11. The method of claim 9, wherein at least some of the N-glycolylneuraminic acid or derivative thereof is bound, and the composition further comprises a catalytic amount of at least one pharmaceutically acceptable acid or a salt thereof, and at least some of the acid or salt thereof is dissolved by saliva in the patient's mouth.

12. The method of claim 1, wherein the dosing regimen comprises administering the composition at least once per day.

13. The method of claim 1, wherein the dosing regimen comprises administering to the patient between about 0.1 mg and 1000 mg of the composition per day.

14. The method of claim 1, wherein the dosing regimen comprises administering to the patient between about 0.2 mg and 100 mg of the composition per day.

15. The method of claim 1, wherein the dosing regimen comprises administering to the patient between about 0.2 mg and 80 mg of the composition per day.

16. The method of claim 1, wherein the viral hepatitis infection comprises at least one of hepatitis A virus, hepatitis B virus, hepatitis C virus, hepatitis D virus, hepatitis E virus, or a non A-E hepatitis virus.

17. The method of claim 1, wherein the viral hepatitis infection comprises hepatitis C virus.

18. The method of claim 1, wherein the composition is administered in addition to at least one of an antiviral medication, a medication to alleviate or reduce a symptom of the hepatitis infection, or a medication to alleviate or reduce a side effect of a drug being administered to the patient.

19. The method of claim 1, wherein the composition is administered as a prophylactic to the patient, and wherein the method comprises administering the composition to the patient in an amount sufficient to prevent viral hepatitis infection.

20. The method of claim 1, wherein the composition comprises between about 0.002 wt % and 20 wt % of the N-glycolylneuraminic acid or the derivative thereof.

21. The method of claim 1, wherein the composition comprises between about 0.1 wt % and 20 wt % of the N-glycolylneuraminic acid or the derivative thereof.

22. The method of claim 1, wherein the composition comprises between about 0.1 wt % and 10 wt % of the N-glycolylneuraminic acid or the derivative thereof.

23. The method of claim 1, wherein the composition comprises N-glycolylneuraminic acid.

24. The method of claim 1, wherein the composition comprises a derivative of N-glycolylneuraminic acid.

25. The method of claim 1, wherein the composition comprises a phosphorylated N-glycolylneuraminic acid.

26. The method of claim 1, wherein the composition comprises a sulfated N-glycolylneuraminic acid.

27. The method of claim 1, wherein composition comprises synthetic N-glycolylneuraminic acid.

28. The method of claim 1, wherein composition comprises N-glycolylneuraminic acid extracted from a biological sample.

29. The method of claim 1, wherein the composition comprises a biological sample comprising the N-glycolylneuraminic acid or the derivative thereof

30. The method of claim 1, wherein the composition is in the form of a tablet, a lozenge, a sucker, a semi-soft candy, a gum, a gel, a paste, a mouthwash, or a film.

31. The method of claim 1, wherein the composition further comprises at least one of a coloring agent, a polypeptide, a lubricant, a coating, a sweetener, a flavoring, an antibacterial agent, a taste modifier, a preservative, a disintegrator, a disintegration-preventor, a binder, an antioxidant, a dietary supplement, an antiblocking agent, an antisticking agent, an absorption promoter, an absorption-adsorption carrier, or a therapeutic agent that is pharmaceutically acceptable.
